(19) United States
(12) Patent Application Publication
(10) Pub. No.: US 2004/0170666 A1
(43) Pub. Date: Sep. 2, 2004

(54) Eye coverings

(76) Inventors: Richard Keates, Solebury, PA (US); Ronald Alan Coffee, Surrey (GB); Jonathan Essex-Lopresti, Berkshire (GB); David Neville Davies, Oxford (GB); Margaret Sin Ka Wan, Oxford (GB); Marie Pollard, Ontario (CA)

Correspondence Address: Frost Brown Todd, 2200 PNC Center, 201 East Fifth Street, Cincinnati, OH, 45202, US

(21) Appl. No.: 10/477,645

(22) PCT Filed: May 14, 2002
(86) PCT No.: PCT/GB02/02240

(30) Foreign Application Priority Data
May 14, 2001 (GB) 0111721.7

(51) Int. Cl. A61F 2/00

(52) U.S. Cl. 424/428

(57) ABSTRACT

An eye covering (700) for covering an exposed surface portion of an eye, the covering comprising a body having front and rear surfaces (702a and 701a) with the rear surface being shaped to conform to said exposed surface portion (701, 702) and at least a portion of the body comprising biologically compatible polymer fibre.

This invention relates to eye coverings in the form of contact lenses or eye bandages especially, but not exclusively, for use during and after eye surgery.

In recent years, surgical techniques have been developed that enable the refractive errors in the eye to be corrected by using an excimer laser to ablate corneal tissue. One way of carrying out this procedure is known as photo refractive keratectomy (PRK). PRK treats refractive errors by removal of tissue from the surface of the cornea so as to enable the eye to focus light more directly on the retina. The PRK procedure begins with topical delivery to the eye of anaesthetic followed by removal of a precise amount of corneal tissue by using an excimer laser to ablate or vaporise the part of the corneal tissue to be removed. Because this procedure involves removal of some of the corneal epithelial layer, patients can experience considerable pain until the epithelium heals and covers the treated area. Although eye drops containing pain killers and antibiotics are effective in reducing both operative pain and risk of infection, it is very difficult to control dosage and it is usual for over or under application to occur which may detrimentally affect the healing time.

In PRK the epithelial layer regenerates over the cornea after surgery within about three days and reasonable vision usually results within about seven days. In an attempt to reduce pain and healing time, other techniques have been developed in which, instead of removing the epithelial layer, the epithelial layer is treated with alcohol pealed back as a hinged flap to expose the underside of the cornea for laser ablation and then laid back. This procedure is more comfortable then PRK but is cumbersome and has a slower visual recovery.

In one aspect, the present invention provides a method of delivering an active ingredient such as an anaesthetic, an antibiotic or analgesic, an anti-bacterial, an anti-viral, an anti-fungal antibiotic such as chloramphenicol, gentamicin and ciprofloxacin or analgesic such as aspirin, ibuprofen, paracetamol to an exposed surface portion of an eye before, during or after laser eye surgery such as photo refractive keratectomy or after trauma, which method comprises subjecting liquid carrying the active ingredient to be delivered to the exposed surface portion of the eye to an electric field that causes the liquid to form a jet which thereafter forms at least one fibre or breaks up into fibre fragments or droplets which deposit onto the eye, thereby delivering the active ingredient to the eye.

This method of processing liquid is known as electrohydrodynamic processing (EHD) and is described in, for example, GB-A-1569707, WO 98/03267 and WO 00/67694, and enables good control over the amount of active ingredient delivered. The electrically charged nature of the resultant fibre, fibre fragments and/or droplets, (hereinafter referred to individually or collectively as “comminuted matter”) results in a very even distribution of comminuted matter over the exposed surface portion of the eye, so allowing the active ingredient carried by the comminuted matter to be evenly distributed over the exposed surface portion and therefore enabling a smaller dose of active ingredient than otherwise would be the case to be delivered to the exposed surface portion. In addition, the concentration of the active ingredient within the liquid can be well controlled and starting and stopping of the electrohydrodynamic process can be precisely defined. All of these factors mean that using electrohydrodynamic processing enables active ingredient to be delivered gently and uniformly to the exposed surface of the eye and with a precise dosage because nearly 100% efficiency in transfer of the comminuted matter to the exposed surface portion of the eye can be achieved.

The active ingredient delivered to the exposed surface portion of the eye by the electrohydrodynamic processing may be, for example, a pain killer for reducing post-operative pain, an antibiotic for reducing risk of infection, a growth factor such as hepatocyte growth factor to promote epithelial cell growth during the healing process or a phospholipid surfactant such as a surfactant protein or lecithin which may also enhance the healing of the epithelial layer. Other factors may be delivered to reduce inflammation. Electrohydrodynamic processing may also be used for the delivery of active ingredient in the form of drugs medicaments, biological molecules and the like to exposed surface portions of the eye for non-trauma or non-surgical applications, for example, in the treatment of eye diseases such as glaucoma.

In another aspect, the present invention provides an eye covering manufactured by electrohydrodynamic processing and adapted to make contact with an exposed surface portion of an eye, for example after trauma or before, during or after surgery, for example, laser eye surgery such as PRK. Such an eye bandage may be used to reduce discomfort and pain caused by exposing cut nerve endings during the surgery and also to prevent further damage to the epithelial layer after the laser surgery. The eye bandage may carry an active ingredient, such as one of the active ingredients mentioned above for delivery to the exposed surface portion of the eye.

In one aspect, the present invention provides a method of forming an eye bandage in situ, which method comprises using electrohydrodynamic processing to spray onto the exposed surface portion of an eye, fibres, droplets, fibrils (that is short fibre length or fragments) or combinations thereof of biologically compatible natural or synthetic polymers so as to form a thin translucent layer directly on the exposed surface portion.

Typically, the comminuted matter will have a diameter in the range of 1 to 10 micrometers although diameters of upto several hundred micrometers may be used.

Usually, droplets, fibre and fibrils will be generally circular in cross-section but, if not, the diameter range should be taken as the equivalent circle diameter, that is the diameter of a circle having the same area as the fibre cross-section.

In one embodiment, the method involves-electrohydrodynamic processing of a biologically compatible polymer to spray at least polymer fibres or fibrils onto the exposed surface portion of the eye. The biologically compatible polymer may be a biodegradable polymer such as polylactide having a half-life comparable to the time of normal healing so that there is no need to remove the fibre bandage.

In another aspect, the present invention provides a pre-formed eye covering for covering an exposed surface portion of an eye, the covering comprising a body having front and rear surfaces with the rear surface being shaped to conform to the exposed surface portion of the eye and at least a portion of the body comprising biologically compatible polymer fibre which may, for example, carry an active ingredient to be delivered to the eye such as, for example, an antibiotic to reduce the risk of infection, a pain killer to reduce pain, a growth factor such as hepatocyte or epidermal growth factor to promote epithelial cell growth during the healing process, and/or a phospholipid surfactant such as a surfactant protein which may also enhance the healing of the epithelial layer. Again, the use of EHD processing to produce the biologically compatible polymer fibre enables precise control and distribution of the active ingredients so enabling precise control of the dosage delivered to the eye. Moreover, in this case, the eye covering can be pre-formed so that it is not necessary for the surgeon to spray electrohydrodynamically processed comminuted matter directly into the patient's eye.

The body of the eye covering may comprise a conventional contact lens onto which the biologically compatible polymer fibre may be sprayed by the surgeon immediately prior to use. As another possibility, the biologically compatible polymer fibre may be sprayed onto the contact lens after manufacture and immediately before packaging of the contact lens in a hematically sealed package.

The polymer may be selected from, for example, collagen, polylactide, 2-hydroxyethylmethacrylate and polyethylene oxide.

In one aspect, the present invention provides an eye covering for covering an exposed surface portion of an eye, wherein the covering comprises a plurality of layers of different polymer fibre one of which comprises a hydrophilic polymer fibre such as a hydrogel for example 2-hydroxyethylmethacrylate (HEMA). At least one other layer of the polymer fibre may comprise water-based polymer fibres such as, for example, collagen, chrondroitin sulfate, gelatin, gum arabic, hydroxypropylcellulose, hydroethylcellulose, hydroxypropyl methyl cellulose, carboxymethyl cellulose, Eudragit S100™ (a co-polymer of methyacrylic acid and methylmethacrylate), polyvinyl alcohol, polyvinyl pyrrolidone, 2-hydroxyethylmethacrylate, polyethylene oxide (PEO). The water-based polymer fibre layer may be provided on a front or rear surface of the eye covering. A water-soluble polymer fibre layer may be designed to dissolve or disintegrate instantly in the presence of tears so leaving the hydrophilic polymer fibre layer. The hydrophilic polymer fibre layer may be prehydrated to form a hydrogel which may be cross-linked by ultra-violet or chemical reaction before application of the water-soluble polymer or may hydrate becoming a hydrogel when the eye covering is applied to the exposed surface portion of the eye. The hydrophilic fibre layer may be configured to provide at least some rudimentary optical properties similar to those of a conventional contact lens. The presence of the water-soluble polymer fibre layer facilitates easy handling of the eye covering.

In one aspect, the present invention provides an eye covering for contacting an exposed surface portion of an eye, for example the epithelial layer, which eye covering comprises a plurality of layers of polymer fibre with one of the layers of polymer fibre comprising layers of a hydrophilic polymer such as HEMA or a water-soluble polymer fibre such as polyvinyl pyrrolidone (PVP) or polyvinyl alcohol (PVA). Another layer of polymer fibre may comprise polymer fibres of a biodegradable non-aqueous based polymer, for example, polylactide, polyglycolide, polylactide-co-glycolide, polycaprolactone, polylactide-co-caprolactone, Eudragit™ (a co-polymer of acrylic and methyacrylic acid esters), Biopol (a co-polymer of hydroxybutyrate and hydroxyvalerate).

An eye covering embodying the invention may comprise or include fibres of a non-biodegradable non-aqueous based polymers, for example, polyvinyl acetate, nitrocellulose, polyvinylchloride. In this case, the eye covering will need to be removed in due course.

As another possibility, an eye covering for contacting an exposed surface portion of an eye may be formed from polymer fibres consisting of one or more types of polymer, natural or synthetic, for example any one or more of the polymers mentioned above.

The polymer fibre layers such as HEMA and PEO fibre layers may provide adhesive layers that facilitate adhesion to the eye while the non water-soluble polymer fibres mentioned above should provide increased strength during use.

The polymer fibre may carry an active ingredient (within the polymer fibre or sprayed onto the polymer fibre) to be delivered to the eye enabling, as described above, precise targeted delivery of a medicament or other material to the eye.

The active ingredient may be any of the ingredients mentioned above such as a pain killer or analgesic, an antibiotic, an anti-inflammatory, another medicament, a biological molecule such as a growth hormone and so on. Where the active ingredient is incorporated into the fibre, then the liquid formation from which the fibre is produced may comprise a fully dissolved solution of the active ingredient, a suspension or a microemulsion, for example.

The use of polymer fibre to form the eye covering has further advantages in that the strength-to-weight ratio of the eye covering should be greater than that of a conventional eye bandage or contact lens. Thus, the polymer fibre eye covering may be made very thin for example less than 200 micrometers thick, typically 15 to 70 micrometers thick, and this, combined with the relative porous nature of the fibre network, means that the eye covering is very light in comparison to a conventional contact lens and should therefore stay in position on the exposed surface portion of the eye better than would a conventional contact lens. Indeed, such an eye covering may be sufficiently light in weight that pre-shaping to conform to the exposed surface portion may not be necessary in order for the eye covering to stay in place.

Typically, the eye covering will be circular having a diameter sufficient just to cover the injured epithelial layer and cornea for example 9 to 15 mm (just enough to cover the epithelial layer which is about 8 mm in diameter). Larger or smaller eye coverings may, however, be provided, where appropriate.

In one aspect, the present invention provides an eye covering for covering an exposed surface portion of an eye, wherein the covering comprises a body formed of biologically compatible polymer fibre that is also biodegradable so that, when the eye covering is used after eye surgery, the eye covering degrades or disintegrates in a time comparable with the time of normal healing, typically two to three days for PRK laser surgery.

The present invention also provides an eye covering as set out above hematically sealed within a blister pack or capsule.

In another aspect, the present invention provides a conventional contact lens such as a hydrogel contact lens, wherein at least a proportion of the contact lens for example the periphery, is structurally reinforced by a thin coating of polymer fibre deposited onto the contact lens using EHD processing. As used herein the term “hydrogel” refers to a water soluble (hydrophilic) polymer that maintains some sort of solid structure when exposed to water. Such reinforcement of the contact lens should reduce the possibility of damage, in particular should increase the resistance of the contact lens to shear tearing, and so should facilitate handling during use. The thin coating may be of a water-soluble polymer fibre that dissolves in the eye so that, once the contact lens has been correctly positioned in the eye, the reinforcement disintegrates or dissolves, enabling the contact lens proper to be thinner than is usually possible which should provide greater comfort in use. As another possibility the reinforcing polymer fibre may be biodegradable or non-biodegradable.

As used herein the term “biologically compatible polymer” means any polymer, synthetic or natural, that can be applied to or deposited onto an exposed surface portion of an eye without any unintended adverse or any unintended significant adverse effect.

As used herein, the term “active ingredient” includes any material that has an effect, generally not an adverse effect, on the eye or its environment, for example, drugs or medicaments such as analgesics, pain killers, biological molecules and so on.

As used herein the term “biologically degradable polymer” means that the polymer degrades, disintegrates or dissolves when used for its intended purpose, that is when placed on an exposed surface portion of the eye, within a relatively short period of time, for example, a matter of a few days, commensurate with the length of time normally required for healing subsequent to surgery on or trauma to the eye.

Embodiments of the present invention will now be described, by way of example, with reference to the accompanying drawings, in which:

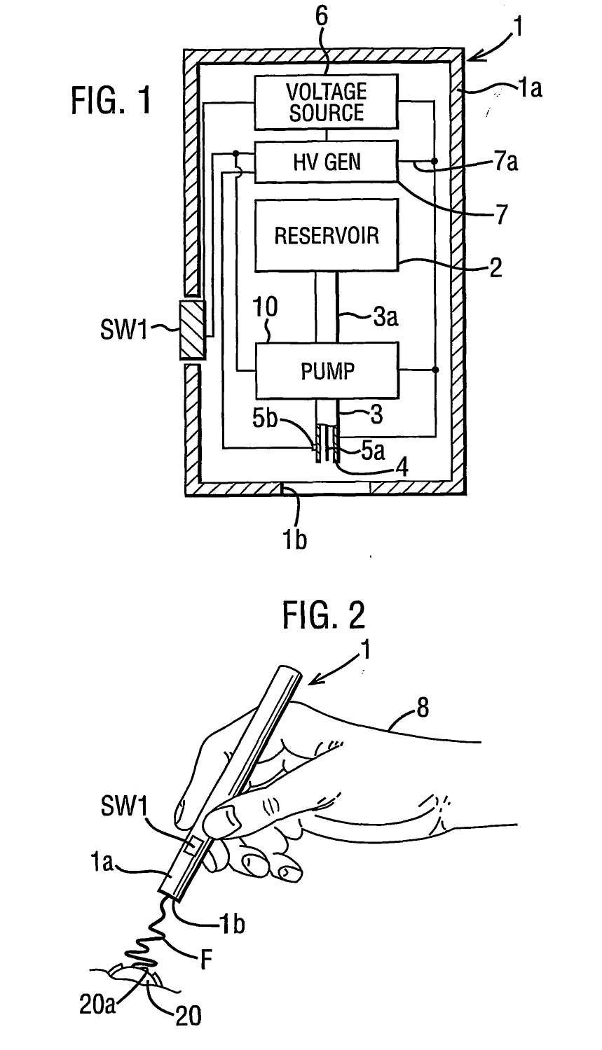

FIG. 1 shows a very diagrammatic cut-away view of an electrohydrodynamic processing device (EHD device);

FIG. 2 shows a diagram illustrating use of the EHD device shown in FIG. 1 to apply comminuted matter to an exposed surface portion of an eye;

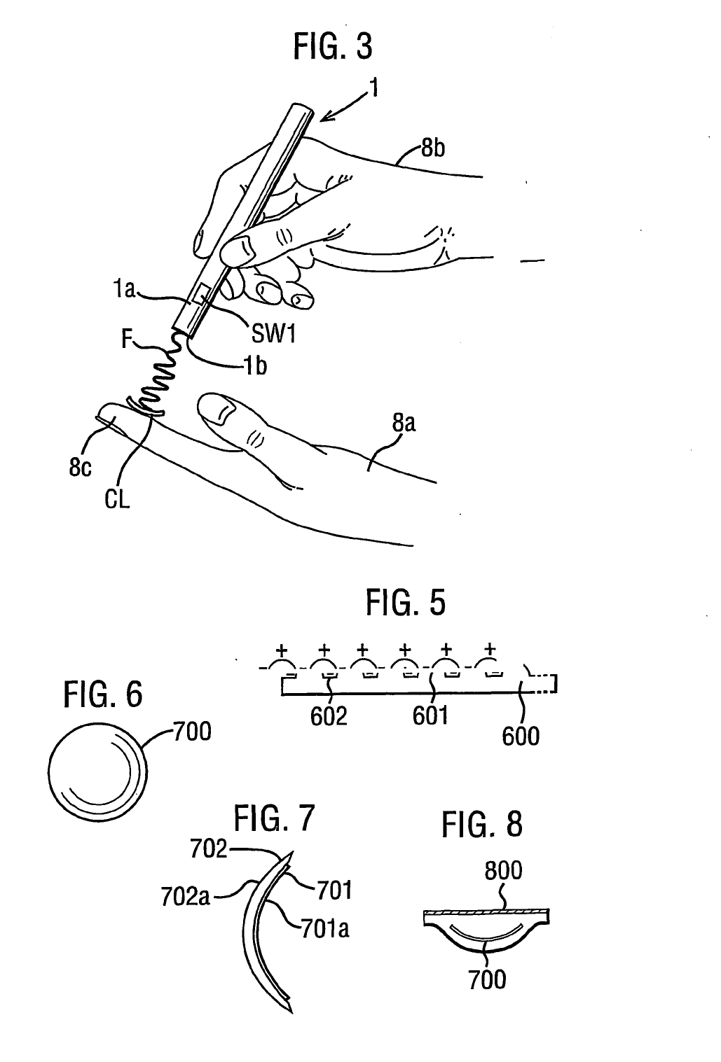

FIG. 3 illustrates diagrammatically use of the EHD device shown in FIG. 1 to deposit comminuted matter onto a contact lens or pre-formed eye bandage;

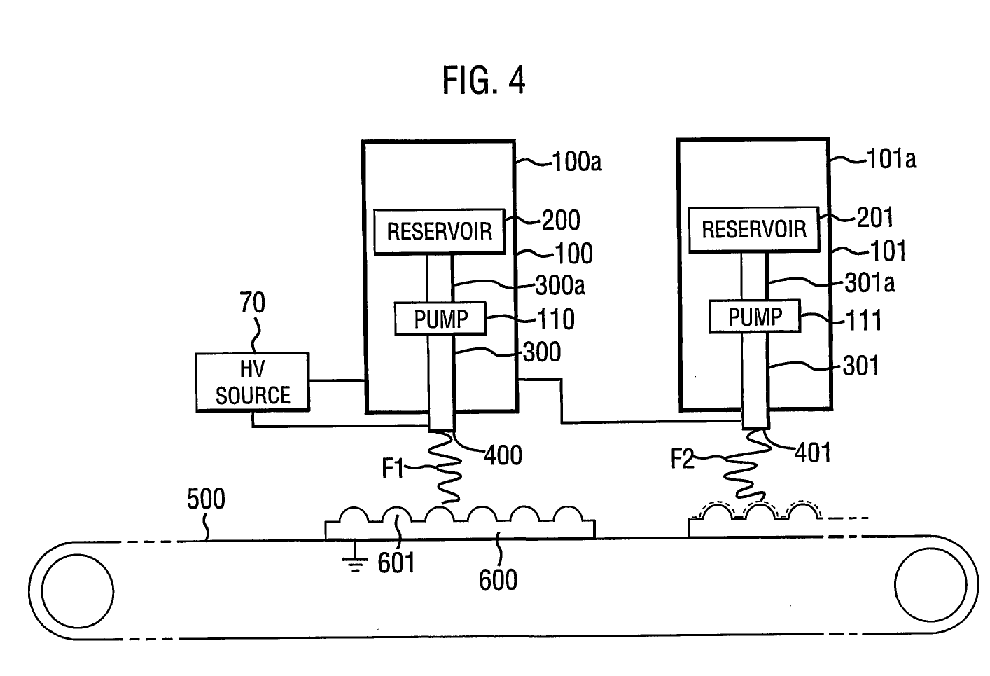

FIG. 4 shows a very simplified diagrammatic representation of electrohydrodynamic processing apparatus for producing eye coverings embodying the present invention;

FIG. 5 shows a very diagrammatic representation of a former suitable for use in the apparatus shown in FIG. 4;

FIGS. 6 and 7 show, very diagrammatically, a front view and a cross sectional view through an eye covering embodying the present invention; and

FIG. 8 shows a cut-away diagrammatic representation of a blister pack incorporating an eye covering embodying the present invention.

Referring now to the drawings, FIG. 1 shows very diagrammatically one example of an electrohydrodynamic processing (EHD) device 1 with a housing 1a of the device 1 cutaway to show functional components of the device.

The housing 1a contains a reservoir 2 of liquid formulation to be subject to EHD processing. The reservoir 2 is coupled via a supply pipe 3a to a pump chamber 10 which is itself coupled to pump liquid into a supply tube 3 having an outlet 4.

In this example, the supply tube 3 is formed of an electrically insulative material. A first electrode 5a is mounted within the electrically insulative tube 3 and a second electrode 5b is mounted to the exterior of the electrically insulative tube 3.

The outlet 4 is positioned adjacent a housing outlet 1b to enable comminuted matter to be dispensed from the EHD device 1 as will be described below.

The housing also contains a voltage source (6 such as a battery), which is coupled via a switch SW1, generally a push button switch mounted to the housing 1a, to a high voltage generator 7 and to the pump 10.

The high voltage generator 7 may be, for example, an electromagnetic high voltage multiplier of the type supplied by Brandenburg, Astec Europe of High Street, Wollaston, Stourbridge, West Midlands DE8 4PG, UK, or Start Spellman of Unit 1, Broomers Park, Broomers Hill Lane, Pullborough, West Sussex, RH20 2RY, UK. As an alternative, a piezoelectric high voltage source which has a low capacitance may be used. Typically, the high voltage generator generates a voltage of several kilovolts for example a voltage in the range of 10 to 20 kilovolts.

In this example, the EHD device 1 is designed so as to be used by an ophthalmic surgeon before, during or after laser eye surgery such as PRK type laser eye surgery and the housing 1 is sized and shaped so as to be grasped easily in the hand.

The reservoir 2 contains a liquid formulation comprising a biologically compatible natural or synthetic polymer carrying an active ingredient.

In this example, the EHD device 1 is intended to be used by the surgeon after PRK or similar laser eye surgery and accordingly the active ingredient comprises at least one of a pain killer, an antibiotic and a growth factor such as hepatocyte growth factor for prompting epithelial cell growth during healing. Other active ingredients that may be incorporated include anti-inflammatories. The manner in which the active ingredient is carried by the liquid formulation will depend upon the particular formulation and active ingredient. For example, the active ingredient may be fully dissolved in the liquid formulation, may be provided in suspension in a liquid formulation or in microsuspension or as the microemulsion within the liquid formulation.

In this example, once the surgeon has carried out the PRK or similar laser treatment, that is the desired amount of corneal tissue has been ablated using an excimer laser, then the surgeon grasps the EHD device 1 in his hand 8 so that, as shown in FIG. 2, the outlet 1b of the housing 1a is positioned over the portion 20a of the eye surface 20 exposed during the laser eye surgery and then activates the switch SW1 using, for example, a finger. Activation of the switch SW1 couples the voltage source 6 to the high voltage generator 7 and the pump 10 so that liquid is pumped to the outlet 4 and liquid issuing from the outlet is subjected to the high electric field generated by the high voltage generator 7 causing the liquid to form a jet. As described with reference to FIGS. 2a to 2c of WO 98/03267 (the whole contents of which are hereby incorporated by reference), dependent upon the liquid formulation and the flow rate to the outlet 4, the liquid jet will form electrically charged comminuted matter which will comprise at least one of electrically charged fibre, fibre fragments (“fibrils”) and droplets. As shown in FIG. 2, the EHD processing results in formation of a fibre F.

Because the comminuted matter is electrically charged, it deposits uniformly and evenly over the exposed surface portion 20a of the eye so enabling uniform delivery of the active ingredient over the exposed surface portion 20a of the eye. In addition, because generation of the comminuted matter can be controlled easily by activating the switch SW1 the surgeon can control accurately the length of time for which comminuted matter is delivered to the exposed surface portion 20a of the eye and can thus control the dose of active ingredient received by the exposed surface portion 20a.

The EHD device 1 thus enables accurate and controlled delivery of an active ingredient to the exposed surface portion 20a to enable reduction of discomfort and pain caused by exposure of cut nerve endings during, for example, PRK laser eye surgery. Where the liquid formulation results in fibre formation as shown in FIG. 2, then the fibres will deposit upon to the exposed surface portion 20a of the eye to build up a thin translucent layer or eye bandage in contact with the exposed surface portion. In this case, in addition to delivering an active ingredient to reduce discomfort and pain, the fibre bandage protects the exposed surface portion 20a against further damage to the epithelial layer and should also provide a physical barrier to reduce the possibility of post-operative infection. while, because of the porous nature of the fibre layer, still allowing air, eye drops and drugs to pass through so that good epithelium health can be maintained. Typically the liquid formulation and flow rate will be selected such that the fibres have a diameter up to several hundred micrometers, preferably in the range 1 to 10 micrometers.

In this example, the liquid formulation comprises a solution of polylactide in a suitable solvent such as acetone or ethyl acetate. The use of polylactide has the advantage that the resultant polymer fibres are biodegradable with a half life comparable to the time of normal healing after PRK laser eye surgery, for example, within two to three days, so that over that time period the fibres gradually disintegrate or dissolve away. This has the advantage that removal of the fibre eye bandage or dressing is not necessary. Other biologically compatible biodegradable polymers may also be used to produce such an in situ polymer fibre eye bandage or dressing such as polyglycolide, polylactide-co-glycolide, polycaprolactone, polylactide-co-caprolactone, Eudragit™ (a co-polymer of acrylic and methyacrylic acid esters), Biopol™ (a co-polymer of hydroxybutyrate and hydroxyvalerate).

In the above described example, the comminuted matter produced by the EHD device 1 is deposited directly onto the exposed surface portion 20a of the eye 20.

FIG. 3 shows a diagram for explaining another form of eye covering or bandage embodying the present invention. In this case, a conventional bandage or contact lens manufactured by a conventional contact lens manufacturing process such as described in, for example, Reports of Patent Design and Trade Mark Cases (RPC) 1997, No. 9 at pages 305, 306 and 350 to 361 is first provided and, as shown in FIG. 3, the EHD device 1 is used to deposit comminuted matter not directly onto the exposed surface portion 20a of the eye but rather onto the rear surface of the contact lens, that is the surface of the contact lens that is placed into contact with the eye during use.

As shown in FIG. 3, in one method, the surgeon places the contact lens CL on a finger tip 8c of one hand 8a (or possibly on another sterile surface) in conventional manner and grasps the EHD device 1 in the other hand 8b so that the outlet 1b of the housing is directed toward the concave or rear surface of the contact lens CL.

When the surgeon then activates the EHD device 1 using the switch SW1, the resulting comminuted matter is deposited directly onto the rear surface of the contact lens CL so depositing fibre, fibre fragments or droplets, as the case may be, containing an active ingredient to applied to the exposed surface portion 20a of the eye onto the rear surface of the contact lens CL.

The surgeon then places the contact lens carrying the active ingredient onto the exposed surface portion 20a of the eye in the normal manner. Again, this allows active ingredient to be evenly applied to the exposed surface portion of the eye in controlled manner. The active ingredient may, again, be at least one of a pain killer for reducing discomfort and pain caused by cut nerve endings exposed during the laser eye surgery, an antibiotic for reducing the possibility of inflammation or infection, a growth factor such as hepatocyte growth factor for promoting epithelial cell growth during the healing process and/or a phospholipid surfactant such as a surfactant protein which may also enhance the healing of the epithelial layer. Also, an anti-inflammatory active ingredient may be delivered.

The comminuted matter may have similar characteristics to those of the comminuted matter discussed above for direct application to the exposed surface portion of the eye.

In the above described examples, comminuted matter is applied directly to an exposed surface portion of an eye or onto a conventional eye bandage or contact lens to enable accurate and controlled delivery of active ingredient to the exposed surface portion of the eye.

A method of manufacturing or preforming an eye covering for delivering active ingredient to an exposed surface portion of an eye will now be described with the help of FIGS. 4 to 8.

FIG. 4 shows a much simplified very schematic diagram of apparatus that may be used in the manufacture of the eye covering. In this case, two EHD devices 100 and 101 are provided. These devices differ from the device 1 shown in FIG. 1 in that they are not intended to be hand held but are intended to be mounted to a gantry or other support (not shown in FIG. 4). Each EHD device 100, 101 comprises a housing 100a, 101a containing a reservoir 200, 201 that supplies liquid formulation via a liquid supply pipe 300a, 301a to a pump 110, 111 coupled to, in this case, an electrically conductive supply tube 300, 301, which projects from an outlet of the housing 100a, 101a of the device. In this case, an external high voltage source 70 is provided that is coupled to each of the electrically conductive tubes 300, 301.

The EHD devices 100, 101 are mounted above an electrically conductive support surface 500 which, in this example, is coupled to earth (ground). As shown, the support surface 500 is in the form of a movable electrically conductive conveyer belt. The surface 500 supports a number of preferably electrically conductive formers 600 each of which defines an array of protrusions 601 each of which is shaped to mimic the surface portion of an eye to which the eye covering is to be applied.

In use, the EHD devices 100 and 101a are activated to produce electrically charged fibre F1, F2 and the movable conveyer belt 500 is moved so as to pass the former 600 beneath first one and then the other of the EHD devices 100 and 101 so that successive layers of polymer fibre F1 and F2 are built up upon the former 600 to form the eye coverings. The high voltage generator may be arranged to cause the fibres F1 and F2 to be oppositely charged so as to facilitate deposition of one upon the other.

The layers of fibre may build up over the entire surfaces of the formers 600, in which case a cutting device such as a knife or laser may be used to separate the individual eye coverings formed on the respective protrusions 601. As another possibility, as shown diagrammatically in FIG. 5 and as described in Wo 00/67694 with respect to FIG. 5 of that document, the formers 600 may be arranged so that the protrusions 601 carry a charge opposite to that of the fibres F1 and F2 while the islands 602 between the protrusions 601 may carry the same charge as the fibres F1 and F2 so that the electrically charged fibres are repelled from the islands 602 and attracted to the protrusion 601. In this case, little or no subsequent separation of the eye coverings should be necessary.

FIGS. 6 and 7 show a front plan view and a cross-sectional view of a resulting eye covering. As can be seen from FIG. 6, the eye covering 700 is generally circular disc-like in shape and, as can be seen from FIG. 7, comprises first and second polymer fibre layers 701 and 702. By virtue of the deposition on the former 601, a rear surface 701a of the eye covering formed by the polymer layer 701 is concave and has a shape that is designed to conform to the exposed surface portion 20a of the eye while the front surface 702a formed the by the polymer fibre layer 702 is outwardly convex.

Generally, the disc-like eye covering will have a diameter comparable to that of a conventional contact lens, for example, 9 to 15 millimetres and may be designed just to cover the injured epithelial layer and cornea.

The fibre layers 701 and 702 may be deposited for a time period such that the overall thickness of the resulting eye covering is less than about 200 micrometers, typically 50 to 70 micrometers.

After manufacture, the eye coverings 700 may be individually hematically sealed in blister packs 800 as shown in FIG. 8 in a manner similar to conventional disposable contact lenses, preserved in an appropriate preserving sterile environment.

Examples of particular liquid formulations for producing particular polymer fibre layers will now be described.

In one example, one of the two polymer fibre layers 701 and 702 of the eye covering 700 is formed of hydrophilic polymer fibre and the other of the two polymer fibre layers is formed of water-based polymer fibres as collagen, chrondroitin sulfate, gelatin, gum arabic, hydroxypropylcellulose, hydroethylcellulose, hydroxypropyl methyl cellulose, carboxymethyl cellulose, Eudragit™ S100 (a co-polymer of methyacrylic acid and methylmethacrylate), polyvinyl alcohol (PVA), polyvinyl pyrrolidone (PVP), 2-hydroxyethylmethacrylate, polyethylene oxide. The liquid formulations used to produce these polymer fibres may consist of the polymer dissolved in a suitable solvent which will depend upon the polymer but may be an aqueous solvent, ethanol or an ethanol water mixture or a melt. Either or both of the liquid formulations may contain any one of more of the active ingredients described above in solution, in suspension, in microsuspension, in emulsion or in microemulsion.

Where, in this example, the second or outer polymer fibre layer 702 is formed of a water-soluble polymer such as PVP or PVA, the purpose of this layer is primarily to strengthen the eye covering to reduce the possibility of damage during the process from removal from the blister pack 800 to placement on the exposed eye portion. In this case, when the eye covering is placed on the exposed eye portion 20a, the presence of tears will cause the water-soluble polymer fibre layer to dissolve virtually instantly leaving the hydrophilic polymer fibre layer in place on the eye. The water-soluble polymer may, upon dissolving, disperse active ingredient into the eye.

The HEMA polymer fibre is used, it may already be hydrated, that is it forms a hydrogel. As another example, the hydrophilic polymer fibre layer may be deposited onto the former 600 in a non-hydrated form. In this case, when the eye covering is placed on the exposed eye portion, the water-soluble polymer fibre layer will again dissolve in the presence of tears and the hydrophilic polymer layer will swell to become a hydrogel that covers the epithelial surface.

As further possibilities, one or other of the fibre layers may be replaced by a polymer fibre layer comprising a biodegradable non-aqueous based polymer, for example polylactide, polyglycolide, polylactide-co-glycolide, polycaprolactone, polylactide-co-caprolactone, Eudragit™ (a co-copolymer of acrylic and methacrylic acid esters), Biopol™ (a co-polymer of hydroxybutyrate and hydroxyvalerate) and/or a non-biodegradable non-aqueous, aqueous based polymer, for example polyvinyl acetate, nitrocellulose, polyvinyl chloride. In each case, the liquid formulation will use a suitable solvent or may comprise a melt if a suitable solvent is not available.

In the above described examples, where a water-soluble polymer fibre layer is provided it forms the outermost layer of the eye covering. However, because the fibre layers are very thin, both the first deposited and the second deposited fibre layer will closely follow the shape of the former 601. Accordingly, the water-soluble polymer fibre layer may provide the rear surface 701a of the eye covering that contacts the exposed surface portion 20a in use, facilitating adhesion of the eye covering to the exposed surface portion.

As a further possibility, such an eye covering may comprise three of more polymer fibre layers, one of which may be a hydrophilic polymer layer and one of which may be a water-soluble polymer layer with the latter acting primarily to strengthen the eye covering before use and during application and possibly also providing a source of active ingredient to be dispersed onto the eye. One or more further ones of the polymer fibre layers may be biodegradable so that the fibres degrade or dissolve gradually with time slowly releasing the same or a different active ingredient to enable controlled or delayed delivery of the same or different active ingredient. As another possibility, the entirety of the eye covering may be formed of biodegradable polymer fibre that has a half life comparable with the healing process so that the eye covering disintegrates or dissolves with time and it is not necessary to remove the eye covering at the end of the healing period.

As another possibility, the eye covering 700 may be formed of fibres of a single type of biodegradable non-aqueous based polymer, for example polylactide, polyglycolide, polylactide-co-glycolide, polycaprolactone, polylactide-co-caprolactone, Eudragit™ (a co-copolymer of acrylic and methyacrylic acid esters), Biopol ™ (a co-polymer of hydroxybutyrate and hydroxyvalerate) and/or a non-biodegradable non-aqueous based polymer, for example polyvinyl acetate, nitrocellulose, polyvinyl chloride.

In the above described examples, where water-soluble polymer is used the other polymer fibres or materials forming the eye covering should be sufficiently dry to avoid causing the water-soluble polymer fibres to dissolve or disintegrate prior to placement in the eye.

In the examples described above with reference to FIGS. 4 to 8, the entirety of the eye covering or bandage is formed of polymer fibres and active ingredient such as those described above may be incorporated into any one or more of the polymer fibre layers. As another possibility, droplets carrying active ingredient may be sprayed onto the polymer fibres during or after the deposition as described in, for example, WO 98/03267 or WO 00/67694.

In the above described examples, the polymer fibres are produced by EHD processing and deposited onto a surface, be it an existing contact lens or a support surface of former. Active ingredient may be sprayed onto the fibre as it is formed or deposited. In addition, the fibre and/or the polymer may be modified during flight or after deposition. For example, the polymer may be a side-chain modified polymer which is cross-linkable by ultra violet (UV) light, in which case the fibre may be exposed to UV light during flight and/or after deposition. Other forms of such processing may be used. For example, the polymer fibre may be subjected to a chemical environment, for example a gaseous environment, that causes a change, such as cross-linking, in the polymer fibre during the flight or after deposition. Where the polymer fibre is subject to modification after the deposition, then part of the deposited fibre may be masked. This would enable, for example, the shape of the eye covering to be defined by masking the deposited fibre and exposing only an unmasked area to a cross-linking or other modifying environment.

The unmodified polymer could then be selected removed, for example washed away, leaving an eye covering of the desired shape.

As another possibility, droplets may be sprayed onto a deposited fibre layer using, for example, EHD spraying that reacts with the deposited fibres to cause hardening or cross-linking of the fibres only in a circular pattern area onto which the droplets are deposited. Again, the areas of the fibre mat onto which the droplets have not been deposited could be selectively removed to leave the desired eye covering shape.

Of course, the above described masking procedures could be reversed, that is the undesired portion of the fibre layer could be modified by reaction with UV light, vapour, gas or droplets of another material to form a reaction product that is easily selectively removed.

In the above described examples, one or more layers of a single type of polymer fibre are used to form the eye bandage or covering. As another possibility, one or more layers may be formed by simultaneous deposition of EHD produced polymer fibres formed of different polymers so that the or each layer of the eye covering or bandage consists of more than one polymer. For example, a layer may include different polymers selected for different characteristics such as strength, water-solubility and hydrophilic nature etc. Relative motion may be effected between the EHD processing devices and the target surface to enhance intermingling of the different fibres.

In the above described examples, the polymer fibre eye coverings are deposited onto a former 600 so that the eye coverings are shaped to conform to the exposed surface portion of the eye. Because the polymer fibre eye coverings are very thin and very light weight (because of the gaps or spaces defined by the fibres during deposition to form the fibre layers), such eye coverings are far less likely to move around on the eye than conventional, relatively massive contact lenses. Accordingly, such eye covering should stay in position better than conventional contact lenses or eye bandages. Further, because of the lightness and thinness of the polymer fibre eye coverings, it may not be necessary to shape the rear surface of the eye covering to conform to the exposed surface portion of the eye and, for example, it may be possible to provide the eye coverings as flat, usually disc-like sheets that when applied to the eye adapt themselves to the surface of the eye. In this case, the former 600 shown in FIG. 4 may be omitted and the fibres deposited directly onto the surface 500 and a cutting device then used, as described in WO 00/67694, to separate the resultant fibre mat into individual eye coverings.

As discussed above, because the eye coverings are so thin, it may not be necessary for the eye covering to be shaped to the eye, particularly where the eye covering has a hybrid structure, that is it is comprised of two or more different polymers. For example, where the eye covering comprises one polymer that absorbs water and forms a hydrogel, the bonds among the structural fibres that gives the dressing its shape should loosen with hydration and therefore the entire dressing may be stored as a rigid flat element and either hydrated just prior to placement on the eye or hydrated with eye drops or natural tears when placed on the eye.

An eye covering embodying the invention may be applied to the eye prior to surgery to prepare the patient for surgery, for example such an eye covering may contain or carry an anaesthetic or numbing agent. This would have an advantage over prior conventional eye bandages in that a precise controlled delivery of the anaesthetic over the entire surface of the eye would be possible.

As mentioned above, non biodegradable hydrophobic polymer fibres such as PVC fibres may be used as the eye covering and then removed after a period by the surgeon.

In the above described examples, when the eye covering 700 is encapsulated in, for example, a blister pack or similar capsule, it may simply be hermetically sealed in sterile air. However, it will generally be desirable to incorporate some form of sterile retaining medium within the blister pack to prevent the eye covering 700 drying out, as is done for conventional disposable contact lenses. Where the polymer fibre eye covering includes a water-soluble polymer fibre layer then the conventional saline solution may be replaced by an appropriate liquid or other sterile environment such as a gaseous environment within which the water-soluble polymer fibre layer will not dissolve. Where is it not required that the water-soluble polymer dissolves or disintegrates in tears, then the polymer may be cross-linked, for example by exposure to ultra-violet or a chemical that induces cross-linking during fibre formation or after deposition.

In this case saline solution may be used. In the above examples described with reference to FIGS. 4 to 8, the entirety of the eye covering 700 is formed from polymer fibre layers and any active ingredient is incorporated into the polymer fibre. As another possibility as described in WO 00/67694, for example, the apparatus shown in FIG. 4 may comprise one or more further EHD devices designed to generate comminuted matter in the form of droplets or fibrils carrying active ingredient which is deposited onto the fibre during or after deposition so that the droplets or fibrils stick to the fibre.

As a further possibility, a main body of the eye covering 700 may be a conventional contact lens onto which one or more polymer fibre layers as described above are deposited. In this case, the polymer fibre layer may be deposited during manufacture after formation of the contact lens or, as another possibility, the polymer fibre layer or layers may be deposited onto a surface of the mould within which the contact lenses are to be formed.

In the above described examples, the eye covering is intended to protect the eye and/or to supply an active ingredient to the eye after laser eye surgery. The device 1 shown in FIG. 2 may also be used to deliver droplets or fibrils carrying an active ingredient such as an analgesic, to an eye prior to such an operation so as to enable precise control over the delivery and dosage of the analgesic. Such a device may also be used to deliver droplets or fibrils carrying active ingredients for treating eye diseases such as a glaucomas. Further the eye coverings described above may be used to protect the eye and/or deliver active ingredient after other forms of surgical procedure and in other cases were protection of the eye is required, for example, during healing of accidental eye trauma.

The pre-formed or pre-manufactured eye coverings described above are intended primarily for medical use by surgeons or like skilled personnel during treatment of an eye. This being the case, it is not generally necessary for the eye covering to have optical properties that correct the wearer's vision. It may, however, be possible to control deposition of the fibre layers and/or to shape the deposited fibre layers using a conventional lathing technique as described in the aforementioned abstract from RPC 1997, No. 9 to provide the eye covering with at least some rudimentary or crude optical characteristics to assist the wearer's vision during the treatment.

In the examples described above, the eye covering is intended to be used during medical treatment, for example after surgery or trauma to the eye. An EHD device as described above may, however, also be used to deposit polymer fibres onto a conventional contact lens, especially a disposable contact lens, to strengthen the contact lens and make it more resistant to shear tearing during handling by the wearer. For example, a water-soluble polymer material may be deposited around the periphery of the contact lens so that the periphery is strengthened during handling but the water-soluble polymer instantly or rapidly dissolves or disintegrates when in the presence of tears, that is when placed on the eye in normal manner. The water-soluble polymer maybe, any of those discussed above. Such conventional contact lenses may also be strengthened by applying a thin coat of a biodegradable polymer fibre such as polylactide, HEMA and PEO polymer fibres may also act as a gentle adhesive to stop the eye covering moving around.

In the above described examples, the EHD devices have a single outlet nozzle. The EHD device may, however, have a number of outlets or nozzles as described in WO 98/03267 enabling, for example, production of more than one fibre at a time. Also, in the above described examples, the polymer fibres are fibres of a single polymer. This need not necessarily be the case and for example, composite polymer fibres may be produced as described in WO 00/67694 with a core of the polymer fibre having different properties from its coating and possibly also carrying different active ingredients. As an example, an outer coating of the polymer fibre may comprise a water-soluble polymer fibre which disperses rapidly when the eye covering is placed in the eye leaving the polymer fibre core intact.

The methods and eye coverings described above enable electrohydrodynamic processing to be used to deliver healing agents and analgesics to an exposed portion of an eye in controlled manner. As used herein, the terms “exposed portion” and “exposed surface portion” should be taken to include both the portion of the eye surface that is normally exposed, that is the conjunctiva and also a surface exposed by trauma or during surgery, for example a corneal epithelial layer exposed during laser ablation of corneal tissue.

Where an EHD produced eye covering is to be used after surgery or trauma, then it may include at least one of droplets, fibrils and fibres of a biologically compatible polymer which degrade in the environment of the eye (for example, under the action of an enzyme in tears) over a period comparable to the normal healing period after such surgery or trauma.

In one aspect, the present invention enables the use of electrohydrodynamic processing to deliver healing agents and analgesics to damaged eye surfaces and should improve surgical operations by enabling controlled doses to be delivered with uniformity and in a gentle patient-friendly manner.

In one aspect, the present invention enables use of an electrohydrodynamically produced polymer fibre mat as an eye bandage of eye cover which has a better strength-to-weight ratio than a conventional contact lens and, unlike a conventional contact lens can also be used to deliver active ingredient to the eye.

In one aspect the present invention uses electrohydrodynamic processing to modify an existing conventional contact lens or eye bandage, for example by coating them with fibres, fibrils and/or droplets of an active ingredient to be supplied to the eye to enable good dosage control or for example to increase the strength, especially the resistance to shear tearing, of the contact lens.

In one aspect, the present invention enables a composite eye covering or contact lens to be produced one face of which is water-soluble and dissolves in the environment of the eye and the other surface of which is water absorbant and, in the presence of tears or suitable eye drops, dissolves to form a hydrogel lens.

In one aspect, the present invention enables the in situ application of a bandage or eye covering of ultrafine fibres (with, for example, diameters in the range of 1 to 10 microns or up to several 100 microns) of a biodegradable polymer such as polylactic acid which has a half life comparable to the time of normal healing, for example 2 to 3 days. This eye covering may formed in situ on the eye by spraying the polymer fibre directly onto the exposed portion of the eye or may be pre-formed for later application to the eye.

As discussed above, the eye bandage or covering may or may not carry one or more active ingredients.

Droplets of PVA or PVP may be sprayed by EHD processing directly into the eye or onto a conventional eye covering or an eye covering embodying the invention prior to, during or after application to the eye, for example for lubrication purposes.

As mentioned above, the techniques disclosed in WO 00/67694 that enable slow or controlled release of drugs or medicaments may also be used.

Other forms of EHD devices than those described above may be used as described in, for example, GB-A-1569707, WO 98/03267 and WO 00/67694 for example at least in some circumstances the pump may be omitted and a gravity feed used.

Other biologically compatible polymers and polymer formulations than those described above may be used.

1. An eye covering for covering an exposed surface portion of an eye, the covering comprising a body having front and rear surfaces with the rear surface being shaped to conform to said exposed surface portion and at least a portion of the body comprising biologically compatible polymer fibre.

2. An eye covering according to claim 1, wherein the said at least a portion of the body comprises a layer of polymer fibre providing one of the front and rear surfaces of the body.

3. An eye covering according to any one of the preceding claims comprising polymer fibre of a polymer selected from the group consisting of collagen, polylactide, 2-hydroxyethylmethacrylate, and polyethylene oxide.

4. An eye covering according to claim 1 or 2, wherein the body comprises a plurality of layers of polymer fibre.

5. An eye covering according to claim 4, wherein one of the layers of polymer fibre comprises a layer of hydrophilic polymer fibre.

6. An eye covering according to claim 5, wherein the hydrophilic polymer is 2-hydroxyethylmethacrylate.

7. An eye covering according to claim 2, 4, 5 or 6, wherein the or at least one of the layers of polymer fibre comprises water-soluble polymer fibre.

8. An eye covering according to claim 2, 4, 5, 6 or 7, wherein the or at least one of the layers of polymer fibre comprises water-based polymer selected from the group consisting of collagen, chrondroitin sulfate, gelatin, gum arabic, hydroxypropylcellulose, hydroethylcellulose, hydroxypropyl methyl cellulose, carboxymethyl cellulose, a co-polymer of methacrylic acid and methylmethacrylate, polyvinyl alcohol, polyvinyl pyrrolidone, 2-hydroxyethylmethacrylate, polyethylene oxide.

9. An eye covering according to any one of claims 2, 4 to 8, wherein the or at least one of the layer of polymer fibre comprises fibre of a polymer selected from the group consisting of a biodegradable non-aqueous based polymer, for example, polylactide, polyglycolide, polylactide-co-glycolide, polycaprolactone, polylactide-co-caprolactone, a co-polymer of acrylic and methacrylic acid esters, a co-polymer of hydroxybutyrate and hydroxyvalerate.

10. An eye covering according to claim 1 or 2, wherein the body comprises a hydrogel.

11. An eye covering according to claim 1, wherein the body comprises a hydrogel body portion and the polymer fibre portion comprises a polymer fibre layer providing at least one of the front and rear surfaces of the body.

12. An eye covering according to claim 11, wherein the hydrogel body portion comprises a further polymer fibre layer comprising layers of hydrogel fibre.

13. An eye covering according to claim 11 or 12, wherein the polymer fibre layer comprises water-soluble polymer fibre.

14. An eye covering according to claim 11 or 12, wherein the polymer fibre layer comprises a water-based polymer selected from the group consisting of collagen, chrondroitin sulfate, gelatin, gum arabic, hydroxypropylcellulose, hydroethylcellulose, hydroxypropyl methyl cellulose, carboxymethyl cellulose, a co-polymer of methacrylic acid and methylmethacrylate, polyvinyl alcohol, polyvinyl pyrrolidone, 2-hydroxyethylmethacrylate, polyethylene oxide.

15. An eye covering according to claim 1, wherein the body comprises a contact lens and said polymer fibre portion of the body comprises a strengthening portion.

16. An eye covering according to claim 15, wherein the polymer fibre portion is provided on a periphery of the contact lens.

17. An eye covering according to claim 15 or 16, wherein the contact lens comprises a hydrogel.

18. An eye covering according to any one of claims 15 to 17, wherein the polymer is selected from the group consisting of polyethylene oxide, polylactide and 2-hydroxyethylmethacrylate.

19. An eye covering for covering an exposed surface portion of an eye, the covering comprising a body having front and rear surfaces with at least one of the front and rear surfaces of the body comprising hydrophilic polymer fibre.

20. An eye covering according to claim 19, wherein the hydrophilic polymer is 2-hydroxyethylmethacrylate.

21. An eye covering according to claim 19 or 20, wherein the body comprises a plurality of layers of polymer fibre.

22. An eye covering according to claim 21, wherein one of the front and rear surfaces of the body comprises the hydrophilic polymer fibre and the other comprises water-soluble polymer fibre.

23. An eye covering according to claim 22, wherein the water-soluble polymer is selected from the group consisting of polyvinyl alcohol and polyvinyl pyrrolidone.

24. An eye covering for covering an exposed surface portion of an eye, the covering comprising a body having front and rear surfaces with the front and rear surfaces of the body comprising layers of different polymer fibre.

25. An eye covering according to claim 24, wherein one of the layers of polymer fibre comprises a layer of hydrophilic polymer fibre and the other comprises a layer of water-soluble polymer fibre.

26. An eye covering according to claim 25, wherein the hydrophilic polymer is 2-hydroxyethylmethacrylate and the water-soluble polymer is selected from the group consisting of polyvinyl alcohol and polyvinyl pyrrolidone.

27. An eye covering according to claim 24, wherein one of the layers of polymer fibre comprises a layer of hydrophilic polymer fibre and the other comprises fibres of a polymer selected from the group consisting of a biodegradable non-aqueous based polymer, for example, polylactide, polyglycolide, polylactide-co-glycolide, polycaprolactone, polylactide-co-caprolactone, a co-polymer of acrylic and methacrylic acid esters, a co-polymer of hydroxybutyrate and hydroxyvalerate.

28. An eye covering according to any one of the preceding claims wherein the or each polymer is at least one of water-soluble, hydrophilic and biodegradable.

29. An eye covering according to any one of the preceding claims, wherein the polymer fibre is formed by electrohydrodynamic comminution.

30. An eye covering according to any one of the preceding claims, wherein the polymer fibre portion carries at least one active ingredient.

31. An eye covering according to claim 30, wherein the active ingredient is selected from the group consisting of an antibiotic, a painkiller, an anti-inflammatory, a growth factor such as hepatocyte growth factor, a phospholipid surfactant.

32. A package comprising an eye covering in accordance with any one of the preceding claims within a hermetically sealed container.

33. A method of modifying an eye covering in the form of a contact lens or contact eye bandage, which method comprises positioning the eye covering adjacent an electrohydrodynamic comminution device comprising a liquid supply with an outlet and an electric field generator and activating the device so that liquid issuing from the outlet is subjected to an electric field causing the liquid to form at least one electrically charged jet which then forms at least one of electrically charged fibre, fibre fragments and droplets which deposit onto the eye covering.

34. A method according to claim 33, wherein the liquid comprises a polymer formulation.

35. A method according to claim 33 or 34, wherein the liquid carries an active ingredient.

36. A method according to claim 35, wherein the active ingredient is selected from the group consisting of an antibiotic, a painkiller, an anti-inflammatory, a growth factor such as hepatocyte growth factor, a phospholipid surfactant.

37. A method of applying an active ingredient to an eye after eye surgery which method comprises placing a contact lens modified by a method in accordance with any one of claims 33 to 36 onto an exposed surface portion of the eye.

38. A method of applying an active ingredient to an exposed surface portion of an eye prior to, during or after surgery, which method comprises providing an electrohydrodynamic comminution device comprising an electric field generator and a liquid reservoir having a liquid outlet and containing a biologically compatible liquid carrying at least one active ingredient, directing the outlet towards the eye and activating the device so that liquid issuing from the outlet is subjected to an electric field causing the liquid to form at least one electrically charged jet which then forms at least one of electrically charged fibre, fibre fragments and droplets which deposit onto the exposed surface portion of the eye.

39. A method of applying an active ingredient to an exposed surface portion of an eye prior to, during and/or after photo refractive keratectomy, which method comprises providing an electrohydrodynamic comminution device comprising an electric field generator and a liquid reservoir having a liquid outlet and containing a biologically compatible liquid carrying at least one active ingredient selected from the group consisting of an analgesic, a painkiller, an antibiotic, a phospholipid surfactant such as a surfactant protein and a growth factor such as hepatocyte growth factor, directing the outlet towards the eye and activating the device so that liquid issuing from the outlet is subjected to an electric field causing the liquid to form at least one electrically charged jet which then forms at least one of electrically charged fibre, fibre fragments and droplets which deposit onto the exposed surface portion of the eye to supply the active ingredient to the exposed surface portion.

40. A method according to claim 38 or 39, wherein the active ingredient is selected from the group consisting of an antibiotic, a painkiller, an anti-inflammatory, a growth factor such as hepatocyte growth factor, a phospholipid surfactant.

41. A method according to claim 38, 39 or 40, wherein the liquid comprises a polymer formulation.

42. A method according to claim 34 or 41, wherein the liquid formulation comprises one or more of the following polymers, a water-based polymer selected from the list consisting of collagen, chrondroitin sulfate, gelatin, gum arabic, hydroxypropylcellulose, hydroethylcellulose, hydroxypropyl methyl cellulose, carboxymethyl cellulose, a co-polymer of methacrylic acid and methylmethacrylate, polyvinyl alcohol, polyvinyl pyrrolidone, 2-hydroxyethylmethacrylate, polyethylene oxide, a biodegradable non-aqueous based polymer selected from the list consisting of polylactide, polyglycolide, polylactide-co-glycolide, polycaprolactone, polylactide-co-caprolactone, a co-polymer of acrylic and methacrylic acid esters, a co-polymer of hydroxybutyrate and hydroxyvalerate, a non-biodegradable non-aqueous based polymer selected from the list consisting of polyvinyl acetate, nitrocellulose, polyvinylchloride.

43. An eye covering according to any one of claims 1 to 38, wherein the polymer fibre comprises fibres of a non-biodegradable non-aqueous based polymer, for example polyvinyl acetate, nitrocellulose, polyvinylchloride.

44. An eye covering according to any one of claims 1 to 38 or 43, wherein the polymer fibre portion or a layer or layers of polymer fibre comprises polymer fibres of two or more different types or different polymers.
